(19) United States
(12) Patent Application Publication
(10) Pub. No.: US 2017/0164873 A1
(43) Pub. Date: Jun. 15, 2017

(54) MEDICAL VENTILATOR WITH PNEUMONIA AND PNEUMONIA BACTERIA DISEASE ANALYSIS FUNCTION BY USING GAS RECOGNITION

(71) Applicant: TAIWAN CARBON NANO TECHNOLOGY CORPORATION, Miaoli County (TW)

(72) Inventors: Yu-Hsuan Liao, MIAOLI COUNTY (TW); Chia-Hung Li, MIAOLI COUNTY (TW); Chun-Hsien Tsai, MIAOLI COUNTY (TW); Ting-Chuan Lee, MIAOLI COUNTY (TW); Chun-Jung Tsai, MIAOLI COUNTY (TW)

(21) Appl. No.: 15/096,760

(22) Filed: Apr. 12, 2016

(30) Foreign Application Priority Data
Dec. 11, 2015 (TW) 104141669

(51) Int. Cl. A61B 5/08; A61M 16/00; A61B 5/00

(52) U.S. Cl. CPC A61B 5/082; A61B 5/7282; A61B 5/7264; A61M 16/0057; A61M 2230/43

(57) ABSTRACT

A medical ventilator with a pneumonia and pneumonia bacterial disease analysis function by using gas recognition includes a sensor array, a sensor circuit, a stochastic neural network chip, a memory and a microcontroller. The sensor array detects a plurality of gases under test and generates a plurality of recognition signals corresponding to the gases under test. The sensor circuit reads and analyzes the recognition signals to generate a plurality of gas pattern signals corresponding to the gases under test. The stochastic neural network chip reduces a dimension of the gas pattern signals to generate an analysis result. The memory stores gas training data. The microcontroller receives the analysis result, and identifies types of the gases under test according to the analysis result.

### FIELD OF THE INVENTION

The present invention relates to a medical ventilator with a pneumonia and pneumonia bacteria disease analysis function by using gas recognition, and particularly to a medical ventilator capable of real-time and accurately detecting a type of gas and providing a pneumonia and pneumonia bacteria disease analysis function.

### BACKGROUND OF THE INVENTION

A medical ventilator is for a patient who cannot breathe spontaneously to sustain vital signs, and is commonly seen in intensive care units and emergency rooms.

For example, the U.S. Patent Publication No. 2007/0068528 A1 discloses an artificial ventilator for determining a ventilation status of a lung. This disclosure includes: a sensor for measuring a gas concentration in expired gas during a single breath, an analog-to-digital converter (ADC) for obtaining data samples of the gas concentration of the expired gas over a single breath in the time domain, means for selecting a plurality of data samples from the obtained data samples, means for calculating a mean tracing value being sensitive to changes of alveolar dead space on the basis of the selected data samples, and a data processor.

For another example, the Taiwan Utility Patent No. M437177U1 discloses a ventilator capable of displaying a suspended particle concentration level. This disclosure includes a housing and a filtering element in the housing. The housing includes an inlet and an outlet. Air enters the housing from the inlet and is discharged from the exit after suspended particles are filtered by the filtering element. One feature of this disclosure is that, the ventilator capable of displaying a suspended particle concentration level further includes a suspended particle concentration sensor in the housing and between the filtering element and the exit, and a display unit electrically connected to the suspended particle concentration sensor and displaying the suspended particle concentration level sensed by the suspended particle concentration sensor. Thus, the display unit allows a user to learn the quality of air provided by the ventilator, so as to replace or clean the filtering element of the ventilator at appropriate timings.

In the prior art above, only a function of purely providing a critically ill patient to breathe normally and sustaining life is provided. However, during a treatment, a critically ill patient has weaker immunity in a way that chances of respiratory tract and lung infections that may trigger complications are greatly increased. Once the infection occurs, a time-consuming inspection process, e.g., X-ray, blood taking or phlegm ejecting, and further testing are required to learn the type of bacterial infection. Such long testing time may endanger the patient's life.

### SUMMARY OF THE INVENTION

The primary object of the present invention is to solve issues of the prior art. In the prior art, a conventional medical ventilator provides a pure function of allowing a critically ill patient to breathe normally and sustaining life. Once an infection occurs during a treatment, a time-consuming testing time is required to learn the type of bacterial infection in a way that the patient's life is endangered by such long testing time.

To achieve the object, the present invention provides a medical ventilator with a pneumonia and pneumonia bacterial disease analysis function by using gas recognition. The medical ventilator of the present invention includes a sensor array, a sensor circuit, a stochastic neural network chip, a memory and a microcontroller. The sensor array includes a substrate, a heating layer on the substrate, an insulation layer on the heating layer, and a plurality of detection units arranged on the insulation layer. Each of the detection units includes at least one detecting electrode, a separating portion surrounding the detecting electrode, and a sensing reaction film. The detecting electrode includes a first electrode and a second electrode. The first electrode includes a first strip-like electrode, and a first finger-like electrode extending from the first strip-like electrode. The second electrode includes a second strip-like electrode, and a second finger-like electrode extending from the second strip-like electrode. The first finger-like electrode and the second finger-like electrode are alternately arranged. The reaction sensing film is in an accommodating space in the separating portion and in contact with the detecting electrode. The reaction sensing film comes into contact with a plurality of gases under test to produce an electrochemical reaction to cause the detecting electrode to generate a plurality of recognition signals corresponding to the gases under test. The sensor circuit reads and analyzes the recognition signals to generate a plurality of gas pattern signals corresponding to the gases under test. The stochastic neural network chip amplifies differences among the gas pattern signals and reduces a dimension of the gas pattern signals to generate an analysis result. The memory stores gas training data. The microcontroller receives the analysis result, and performs a mixed gas recognition algorithm according to the analysis result to identify types of the plurality of gases under test, categorizes an unknown gas that is not included in the gas training data, and generates a recognition result according to the gas training data.

It is known from the above that, the present invention provides following effects compared to the prior art. The medical ventilator with a pneumonia and pneumonia bacterial disease analysis function provides the pneumonia and pneumonia bacterial disease analysis function using gas recognition. Therefore, in addition to providing a patient with a breathing function, the medical ventilator of the present invention is further capable of early detecting the type of bacterial infection of the respiratory tract and lungs and associated complications of the patient, so as to real-time and accurately treat the symptoms and reduce the threat of the complications on the patient.

### BRIEF DESCRIPTION OF THE DRAWINGS

FIG. 1 is a schematic diagram according to an embodiment of the present invention;

FIG. 2 is a block diagram according to an embodiment of the present invention;

FIG. 3 is a top view of a sensor array according to an embodiment of the present invention;

FIG. 4 is a section view of FIG. 3 along A-A; and

FIG. 5 is a schematic diagram of a detecting electrode according to an embodiment of the present invention.

### DETAILED DESCRIPTION OF THE PREFERRED EMBODIMENTS

Details and technical contents of the present invention are given with the accompanying drawings below.

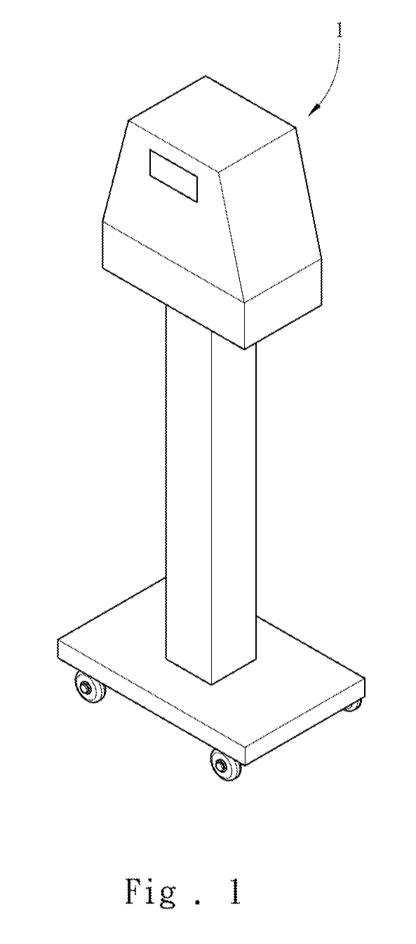

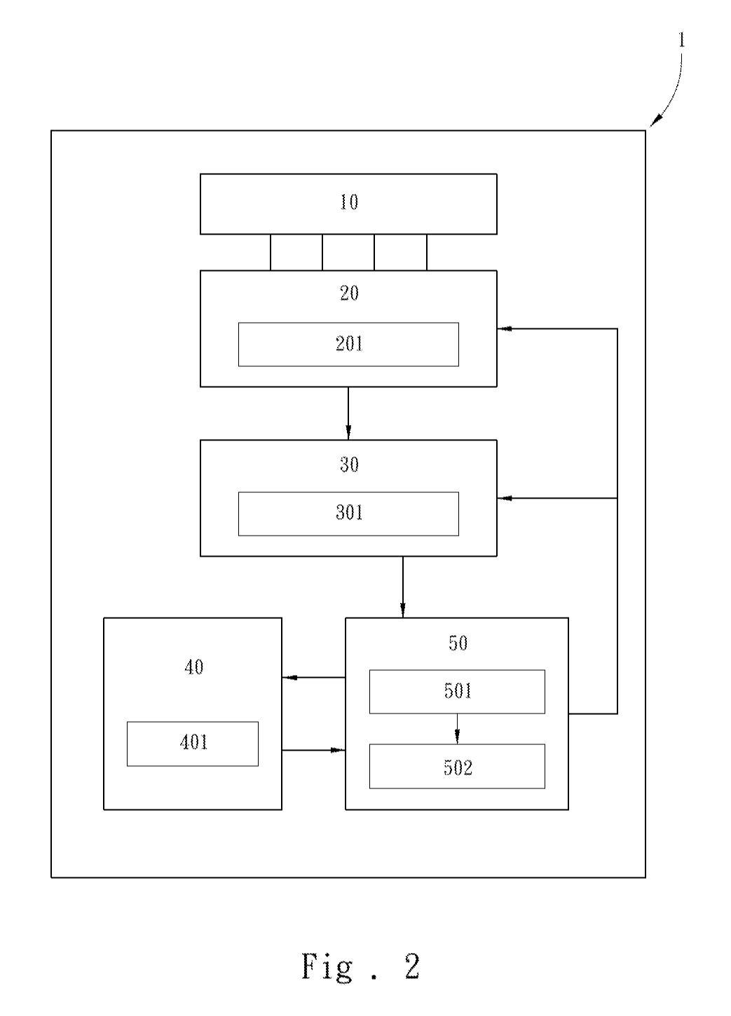

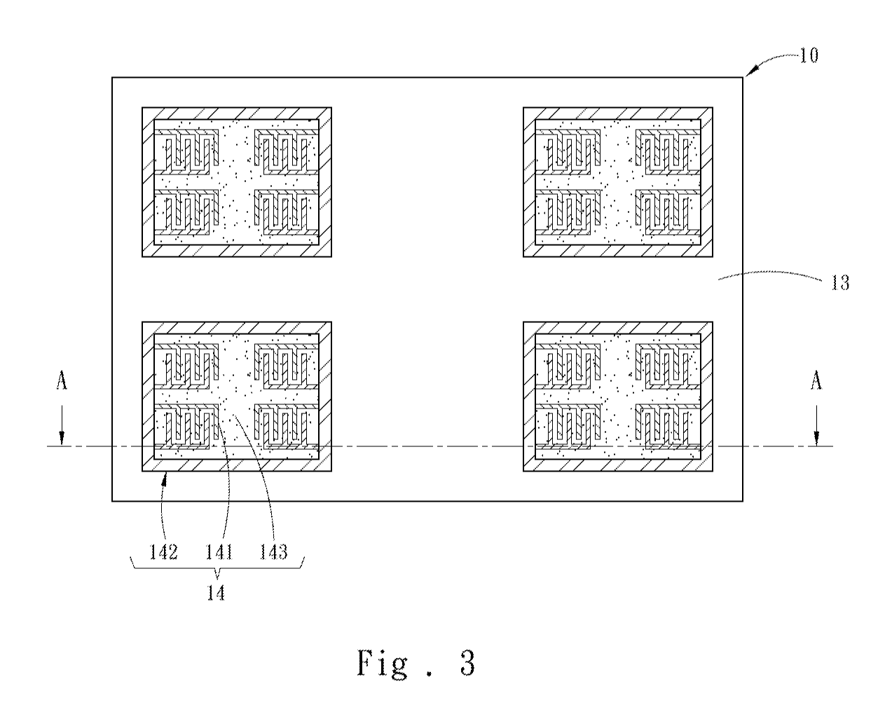

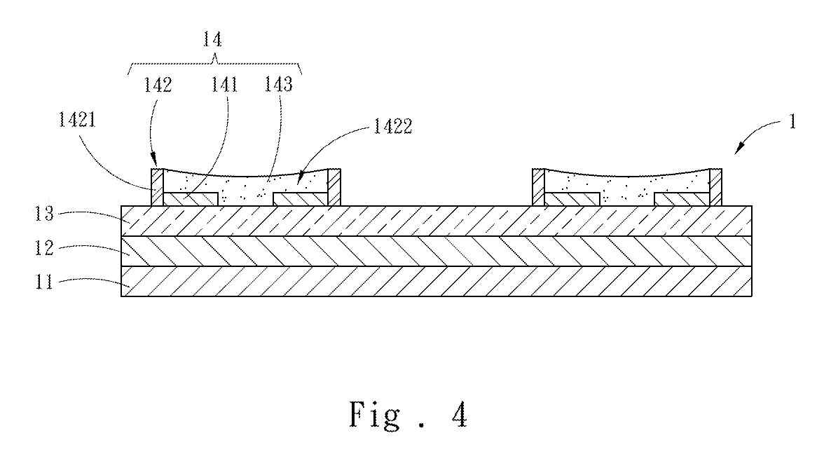

FIG. 1 and FIG. 2 show a schematic diagram and a block diagram of a medical ventilator according to an embodiment of the present invention. Referring to FIG. 1 and FIG. 2, a medical ventilator a with a pneumonia and pneumonia bacterial disease analysis function by using gas recognition includes sensor array 10, a sensor circuit 20, a stochastic neural network chip 30, a memory 40 and a microcontroller 50. FIG. 3 and FIG. 4 show a top view of a sensor array and a section view of FIG. 3 along A-A according to an embodiment of the present invention. Referring to FIG. 3 and FIG. 4, the sensor array 10 includes a substrate 11, a heating layer 12, an insulation layer 13, and a plurality of arranged detection units 14. The heating layer 12 is on the substrate 11. For example, the substrate 11 may be made of a material selected from the group consisting of glass, indium tin oxide (ITO) and polyethylene terephthalate (PET). The heating layer 12 is made of a material that can be heated to a temperature higher than room temperature. In one embodiment of the present invention, the heating layer 12 may be made of ITO, and preferably receives a current and is heated to a temperature between 30° C. and 70° C. The insulation layer 13 is on the heating layer 12, and may be made of PET.

The detection units 14 are on the insulation layer 13, and are arranged in an array or a pattern. In the embodiment, the detection units 14 may be arranged in an 8×4 array, and are preferably spaced by 100 μm from one another. Each of the detection units 14 includes at least one detecting electrode 141, a separating portion 142 and a reaction sensing film 143. In the present invention, the reaction sensing film 143 may be made of at least one material selected from the group consisting of carboxymethyl cellulose ammonium salt (CMC-NH₄), polystyreine (PS), poly(ethylene adipate), poly(ethylene oxide) (PEO), polycaprolactone, poly(ethylene glycol) (PEG), poly(vinylbenzyl chloride) (PVBC), poly(methylvinyl ether-alt-maleic acid), poly(4-vinylphenol-co-methyl methacrylate), ethyl cellulose (EC), poly(vinylidene chloride-co-acrylonitrile) (PVdcAN), polyepichlorohydrin (PECH), polyethyleneimine, beta-amyloid(1-40), human galectin-1 or human albumin, styrene/allyl alcohol (SAA) copolymer, poly(ethylene-co-vinyl acetate), polyisobutylene (PIB), poly(acrylonitrile-co-butadiene), poly(4-vinylpyridine), hydroxypropyl methyl cellulose, polyisoprene, poly(alpha-methylstyrene), poly(epichlorohydrin-co-ethylene oxide), poly(vinyl butyral-co-vinyl alcohol-vinyl acetate), polystyrene (PS), lignin, acylpeptide, poly(vinyl proplonate), poly(vinyl pyrrolidone) (PVP), poly(dimer acid-co-alkyl polyamine), poly(4-vinylphenol), poly(2-hydroxyethyl methacrylate), poly(vinyl chloride-co-vinyl acetate), cellulose triacetate, poly(viny stearate), poly(bisphenol A carbonate) (PC), poly(vinylidene fluoride (PVDF). In the embodiment, the number of the detecting electrodes 141 in each of the detection units 14 may be four, and the detecting electrodes 141 are preferably spaced by 30 μm from one another. As such, the number of the detecting electrodes 141 may be 128. However, the number of the detecting electrodes 141 may be modified according to different application requirements, and is not limited to the example in this embodiment.

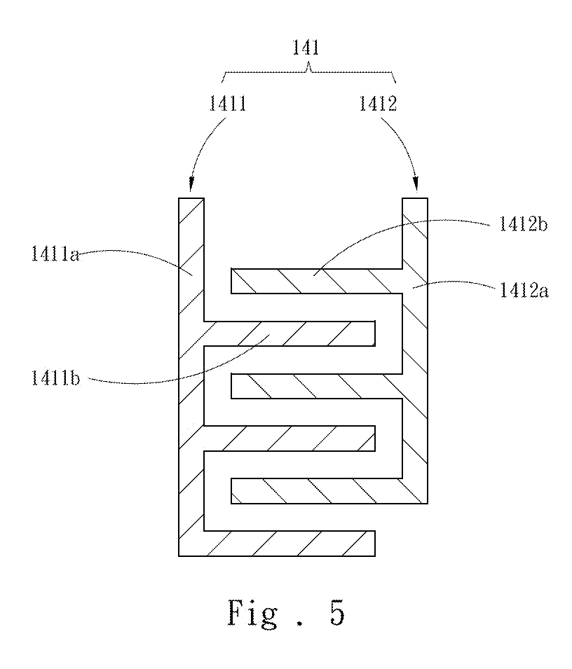

Referring to FIG. 5, each of the detecting electrodes 141 includes a first electrode 1411 and a second electrode 1412. The first electrode 1411 includes a first strip-like electrode 1411a and a first finger-like electrode 1411b. The second electrode 1412 includes a second strip-like electrode 1412a and a second finger-like electrode 1412b. The first strip-like electrode 1411a and the second strip-like electrode 1412a extend along a first axial direction and are parallel. The first finger-like electrode 1411b extends from the first strip-like electrode 1411a towards the second strip-like electrode 1412a along a second axial direction. The second finger-like electrode 1412b extends from the second strip-like electrode 1412a towards the first strip-like electrode 1411a along the second axial direction. The first finger-like electrode 1411b and the second finger-like electrode 1412b are parallel and are alternately arranged. The first axial direction is different from the second axial direction. In the embodiment, the first axial direction is perpendicular to the second axial direction. Further, the detecting electrode 141 may be made of at least one material selected from the group consisting of ITO, copper, nickel, chromium, iron, tungsten, phosphorous, cobalt and silver. The separating portion 142 includes a plurality of separating walls 1421 away from the insulation layer 13 and extending upwards. The separating walls 1421 surround the detecting electrode 141 to form an accommodating space 1422. The reaction sensing film 143 is in the accommodating space 1422 in the separating portion 142 and in contact with the detecting electrode 141. In practice, the reaction sensing film 143 comes into contact with a plurality of gases under test to produce an electrochemical reaction to cause the detecting electrode 141 to generate a plurality of recognition signals corresponding to the plurality of gases under test.

The sensor circuit 20 reads and analyzes the recognition signals to generate a plurality of gas pattern signals 201 corresponding to the plurality of gases under test. According to a collective reaction that the entire array produces for the mixed gases, the sensor array 10 generates the plurality of gas pattern signals 201 corresponding to the gases under test through the sensor circuit 20. The stochastic neural network chip 30 amplifies differences among the plurality of gas pattern signals 201 and reduces a dimension of the plurality of gas pattern signals 201 to generate an analysis result 301.

Further, the stochastic neural network chip 30 may capture main characteristics of the signals by a smart algorithm, and provide an output having a dimension lower than the dimension of the original signals to reduce a computation amount of a backend system. The memory 40 stores the gas training data 401, which includes gas data generated by various bacteria of various complications and other possible gas data. The microcontroller 50 receives the analysis result 301, and performs a mixed gas recognition algorithm 501 according to the analysis result 301 to identify the types of the plurality of gases under test, categorizes an unknown gas that is not included in the gas training data 401, and generates a recognition result 502 according to the gas training data 401.

Further, when the microcontroller 50 detects the unknown gas that is not included in the gas training data 401, the microcontroller 50 automatically categorizes the unknown gas, and transmits unknown gas data corresponding to the unknown gas to the sensor circuit 20, the stochastic neural network chip 30 and the memory 40. As such, the sensor circuit 20 may perform recognition further according to the unknown gas data, the stochastic neural network chip 30 may re-train according to the unknown gas data, and the memory 40 may add one more set of gas training data according to the unknown gas data.

It is known from the above that, the present invention provides following effects compared to the prior art. As the medical ventilator of the present invention includes the gas recognition chip, in addition to providing a patient with a breathing function, the medical ventilator of the present invention is further capable of early detecting the type of bacterial infection of the respiratory tract and lungs and associated complications of the patient, so as to real-time and accurately treat the symptoms.

What is claimed is:

1. A medical ventilator with a pneumonia and pneumonia bacterial disease analysis function by using gas recognition, comprising:
a sensor array, comprising a substrate, a heating layer on the substrate, an insulation layer and a plurality of detection units arranged on the insulating layer, each of the detection units comprising at least one detecting electrode, a separating portion surrounding the detecting electrode, and a reaction sensing film, the detecting electrode comprising a first electrode and a second electrode, the first electrode comprising a first strip-like electrode and a first finger-like electrode extending from the first strip-like electrode, the second electrode comprising a second strip-like electrode and a second finger-like electrode extending from the second strip-like electrode, the first finger-like electrode and the second finger-like electrode alternately arranged, the reaction sensing film in an accommodating space in the separating portion and in contact with the detecting electrode, the reaction sensing film coming into contact with a plurality of gases under test to produce an electrochemical reaction to cause the detecting electrode to generate a plurality of recognition signals corresponding to the plurality of gases under test;
a sensor circuit, reading and analyzing the recognition signals to generate a plurality of gas pattern signals corresponding to the plurality of gases under test;
a stochastic neural network chip, amplifying differences among the gas pattern signals and reducing a dimension of the gas pattern signals to generate an analysis result;
a memory, storing a gas training data; and
a microcontroller, receiving the analysis result, performing a mixed gas recognition algorithm according to the analysis result to identify types of the plurality of gases under test, categorizing an unknown gas that is not included in the gas training data, and generating a recognition result according to the gas training data.

2. The medical ventilator with a pneumonia and pneumonia bacterial disease analysis function by using gas recognition of claim 1, wherein when the microcontroller detects the unknown gas that is not included in the gas training data, the microcontroller transmits unknown gas data corresponding to the unknown gas to the sensor circuit, the stochastic neural network chip and the memory, the sensor circuit performs recognition according to the unknown gas data, the stochastic neural network chip re-trains according to the unknown gas data, and the memory adds one more set of gas training data according to the unknown gas data.

3. The medical ventilator with a pneumonia and pneumonia bacterial disease analysis function by using gas recognition of claim 1, wherein the substrate is made of a material selected from the group consisting of glass, indium tin oxide (ITO) and polyethylene terephthalate (PET).

4. The medical ventilator with a pneumonia and pneumonia bacterial disease analysis function by using gas recognition of claim 1, wherein the heating layer receives a current and is heated to a temperature between 30° C. and 70° C.

5. The medical ventilator with a pneumonia and pneumonia bacterial disease analysis function by using gas recognition of claim 1, wherein the heating layer is made of indium tin oxide (ITO).

6. The medical ventilator with a pneumonia and pneumonia bacterial disease analysis function by using gas recognition of claim 1, wherein the insulation layer is made of polyethylene terephthalate (PET).

7. The medical ventilator with a pneumonia and pneumonia bacterial disease analysis function by using gas recognition of claim 1, wherein the detecting electrode is made of a material selected from the group consisting of indium tin oxide (ITO), copper, nickel, chromium, iron, tungsten, phosphorous, cobalt and silver.

8. The medical ventilator with a pneumonia and pneumonia bacterial disease analysis function by using gas recognition of claim 1, wherein the separating portion comprises a plurality of separating walls away from the insulation layer and extending upwards, and the separating walls surround to form the accommodating space.

9. The medical ventilator with a pneumonia and pneumonia bacterial disease analysis function by using gas recognition of claim 1, wherein the first strip-like electrode and the second strip-like electrode of the detecting electrode extend along a first axial direction and are parallel, the first finger-like electrode extends from the first strip-like electrode towards the second strip-like electrode along a second axial direction that different from the first axial direction, the second finger-like electrode extends from the second strip-like electrode towards the first strip-like electrode along the second axial direction, and the first finger-like electrode and the second finger-like electrode are parallel.

10. The medical ventilator with a pneumonia and pneumonia bacterial disease analysis function by using gas recognition of claim 9, wherein the first axial direction is perpendicular to the second axial direction.
